(19) United States
(12) Patent Application Publication
(10) Pub. No.: US 2008/0249531 A1
(43) Pub. Date: Oct. 9, 2008

(54) Instruments and methods for minimally invasive insertion of dynamic implants

(75) Inventor: Christopher M. Patterson, Olive Branch, MS (US)

Correspondence Address: KRIEG DEVAULT LLP, ONE INDIANA SQUARE, SUITE 2800, INDIANAPOLIS, IN, 46204-2709, US

(73) Assignee: Warsaw Orthopedic, Inc.

(21) Appl. No.: 11/711,421

(22) Filed: Feb. 27, 2007

(51) Int. Cl. A61B 17/58; A61B 17/70; A61B 17/04

(52) U.S. Cl. 606/99; 606/300; 606/96

(57) ABSTRACT

Instruments and methods for dynamic implant placement into a desired location in the patient include a guide instrument that defines a minimally invasive pathway to an implantation location and an inserter instrument guided by the guide instrument to position the dynamic implant along the pathway to the implantation location. The guide instrument can be referenced to the implantation location by mechanical connection or image guided navigation.

### BACKGROUND

The use of prosthetic implants to address orthopedic injuries and ailments has become commonplace. Flexible, non-rigid, and other dynamic implants have been engaged between bony elements and across a joint connecting the bony elements to provide stabilization of the joint while permitting at least limited motion of the bony elements relative to one another. While dynamic stabilization provides benefits in the recovery and outcomes for the patient, there is an ever-present challenge to enable less invasive surgical techniques for placing the dynamic implants between the bony elements, to improve implant delivery capabilities to the surgical site, to shorten the time required to surgically implant dynamic devices, and to decrease surgery recovery time, and provide other improvements. Thus, additional contributions in this area of technology remain desirable.

### SUMMARY

The present invention relates to instruments and methods for dynamic implant placement into a desired location in the patient, and more particularly, but not exclusively, relates to minimally invasive techniques for positioning dynamic implants in a patient. One embodiment includes a unique spinal implantation system and technique for positioning a non-rigid or dynamic implant member in the patient along a percutaneous or other minimally invasive insertion path while minimizing intrusion into the patient anatomy during the procedure. Other embodiments include unique methods, systems, devices, kits, tools, instrumentation, and apparatus involving implantation of a dynamic prosthetic device within a patient.

According to one aspect, a method for inserting a dynamic implant in a minimally invasive procedure comprises: engaging an anchor to a bony element; engaging an elongated extension to the anchor with the elongated extension extending from the anchor to a proximal end; providing a guide instrument including a first portion for manipulating the guide instrument and an elongated second portion extending transversely to the first portion; advancing the second portion of the guide instrument through bodily tissue to a location adjacent the anchor; moving the dynamic implant through a passage of the second portion from a location outside the bodily tissue to the location adjacent the anchor; and engaging the dynamic implant to the anchor.

According to another aspect, an instrument assembly for delivering a dynamic implant in bodily tissue is provided. The assembly includes a guide instrument, a pathway forming instrument removably engagable to the guide instrument for forming a pathway to the implantation location, and an inserter instrument movable through the guide instrument to deliver the dynamic implant to the implantation location in the patient. The guide instrument is configured in one embodiment for pivotal mounting to at least one anchor extension mounted to an anchor engaged to a bony element. In another embodiment, the guide instrument includes a handle for manipulating the guide instrument through bodily tissue.

Related features, aspects, embodiments, objects and advantages of the present invention will be apparent from the following description.

### BRIEF DESCRIPTION OF THE DRAWINGS

FIG. 1 is a schematic elevation view of a spinal column segment with dynamic stabilization systems attached thereto.

FIG. 2 is a schematic elevation view in partial section looking medially-laterally at a spinal column segment with anchors engaged thereto and an anchor extension with an instrument assembly mounted thereto in a first position.

FIG. 3 is the view of FIG. 2 with the instrument assembly in a second position to form a pathway to the anchors.

FIG. 4 is the view of FIG. 3 with a pathway forming instrument removed from the guide instrument of the instrument assembly.

FIG. 5 is an exploded schematic view of an inserter instrument and dynamic implant engageable to the inserter instrument.

FIG. 6 is the view of FIG. 4 with the inserter instrument and dynamic implant positioned in the guide instrument and a second anchor extension engaged to the second anchor.

FIG. 7 is a portion of the view of FIG. 6 showing the dynamic implant extending between the anchors, the distal portions of the anchor extensions in section, and a driving instrument positioned in the anchor extensions to secure engaging members to the anchors.

FIG. 8 is a schematic elevation view in partial section looking medially-laterally at a spinal column segment with anchors engaged thereto, anchor extensions engaged to each anchor, and an instrument assembly mounted to the anchor extensions and moved to form a pathway between the anchors.

FIG. 9 is a schematic elevation view in partial section looking medially-laterally at a spinal column segment with anchors engaged thereto, with anchor extensions engaged to each anchor, and an instrument assembly moved by image guided navigation remotely from the anchor extensions to form a pathway between the anchors.

FIG. 10 is an exploded schematic view of an inserter instrument and dynamic implant positionable between the anchors of FIGS. 8 and 9.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Dynamic implants for implantation in the human body can be desired for stabilization of bony elements, tissue repair, to provide scaffolds between adjacent anatomical elements for structural support and tissue growth, and for other beneficial reasons. Delivery of such implants in a desired configuration and/or at a desired location in the patient's body can be difficult since the implants can alter in form or shape if a trailing end of the implant is pushed during implantation and the leading end of the implant encounters resistance as it is pushed through bodily tissue. Pulling the implants to the implantation location increases invasiveness with additional openings through the tissue of the patient to accommodate instrumentation that forms the pathway from the insertion location to the implantation location and the pathway from the implantation location to the exit location along which the pulling instrumentation is manipulated to position the implant at the implantation location.

Systems and methods for insertion of dynamic implants for connection with one or more anchors engaged to bony elements of the body are provided that minimize the number of openings and intrusiveness into the bodily tissue of the patient while providing a pathway that minimizes or reduces resistance to insertion of the dynamic implant along the insertion pathway. In one form, the systems include at least one anchor extension extending from at least one anchor engaged to at least one bony element of the body. An instrument assembly is mountable to the at least one anchor extension and operable to form a minimally invasive insertion pathway for the dynamic implant from a location remotely positioned from the at least one anchor to a location adjacent to or within the anchor where the dynamic implant can be secured to the anchor. The dynamic implant can be positioned into the patient in minimally invasive approaches while minimizing trauma and surgical risks to the patient and promoting rapid post-operative recovery. However, applications for the instruments in invasive surgeries are not precluded.

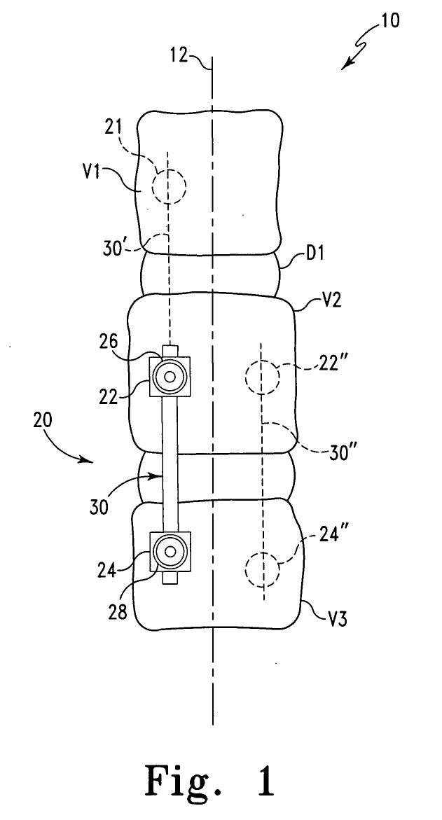

FIG. 1 shows a diagrammatic view of a spinal column segment 10 including three vertebrae V1, V2, V3 and disc space D1 between vertebrae V1, V2 and disc space D2 between vertebrae V2, V3. Spinal column segment 10 extends along a central axis 12, and FIG. 1 is intended to show the posterior side of the spinal column segment 10. Spinal column segment 10 includes a dynamic stabilization system 20 engaged to vertebrae V2, V3 that includes first and second anchors 22, 24 engaged to respective ones of the vertebrae V2, V3 and dynamic implant 30 extending between anchors 22, 24. Dynamic implant 30 is engaged to anchors 22, 24 with respective ones of the engaging members 26, 28. In the illustrated embodiment, dynamic implant 30 is positioned along one lateral side of spinal column segment 10 in an offset relation to central axis 12 and the sagittal plane, which are located medially of dynamic implant 30. In other embodiments, it is contemplated that dynamic implant 30 includes a length sized to extend along three or more vertebrae, as indicated by dynamic implant 30′ in dashed lines that is also secured to vertebra V1 with anchor 21. In still other embodiments, one or more additional spinal implants can be engaged along the vertebrae in other locations, as indicated by dynamic implant 30″ and anchors 22″ and 24″ positioned to form a bi-lateral stabilization system with stabilization system 20 on an opposite side of central axis 12.

In yet another embodiment, dynamic implant 30′ is engaged to vertebrae V1 and V3 with anchors 21 and 24. Anchor 26 is omitted, and two vertebral levels are stabilized with implant 30′ and two anchors engaged to the vertebrae at the ends of the motion segments. One application of such an arrangement is in locations where the distance between the anchoring locations of vertebrae V1, V2 and/or the distance between the anchoring locations of vertebrae V2, V3 is very small.

Anchors 21, 22, 24 can be any suitable anchor for engaging a bony structure such as a vertebral body. In one embodiment, anchors 21, 22, 24 are pedicle screws with a bone engaging portion in the form of a threaded shaft engaged to or through the pedicle of the respective vertebra and a proximal head portion that forms a receiver for engaging implant 30. The receiver can include a U-shaped or other appropriately shaped proximal saddle extending from the threaded shaft. The receiver can include internal and/or external threads for engaging an engaging member such as the respective engaging members 26, 28. The receiver forms a receptacle to receive the portion of dynamic implant 30 positioned therein. The receptacle can be open so that implant 30 can be side-loaded or end-loaded therein from a proximal, medial or lateral side of receiver. In another embodiment, the receptacle is enclosed by the receiver and the receiver includes bores extending into the receptacle for insertion of the implant therethrough in an endwise manner. The receiver can be fixed to the bone engaging portion of the anchor, or movable relative to the bone engaging portion to provide multiple angular arrangements between the receiver and the bone engaging portion.

In spinal surgical procedures, dynamic implant 30 can be delivered to one or more vertebrae or to a disc space between vertebrae in an anterior approach, a posterior approach, a lateral approach, postero-lateral approach, a transforaminal approach, or an anterior-oblique approach, for example. Vertebrae V1, V2, V3 can comprise all or a portion of the cervical, thoracic, lumbar and sacral vertebrae. In addition to stabilization of one or more motion segments with dynamic implant 30, other spinal repair procedures can be performed as an additional procedure, including procedures to fuse vertebrae with one or more implants or bone graft, to replace one or more vertebral bodies, to repair annulus tissue, or to insert artificial disc components, for example.

Dynamic implant 30 can be fabricated from one or more components that are flexible or exhibit at least some flexibility or non-rigidity to permit motion of the stabilized vertebral level or levels. Some examples include extruded components, machined components, molded components, formed components, and milled components. Other examples of implant components include woven fabric tubing, woven and non-woven mesh, or braided or woven structures. Dynamic implant 30 can include any one or more of sheets, tethers, cords, planar members, bands, wires, cables, or any other component capable of forming or being formed into the implant body. In a further form, dynamic implant 30 may be resilient and/or elastic so it can assume various shapes during and after insertion and attachment while exhibiting a tendency to return to its natural form. In yet another form, dynamic implant 3.0 is substantially inelastic so that the shape achieved upon insertion or deformation is maintained.

Dynamic implant 30 can be made from any biocompatible material, material of synthetic or natural origin, and material of a resorbable or non-resorbable nature. Suitable examples of implant material include autograft, allograft or xenograft; tissue materials including soft tissues, connective tissues, demineralized bone matrix and combinations thereof; resorbable materials including polylactide, polyglycolide, tyrosine derived polycarbonate, polyanhydride, polyorthoester, polyphosphazene, calcium phosphate, hydroxyapatite, bioactive glass, collagen, albumin, fibrinogen and combinations thereof; and non-resorbable materials including polyethylene, polyester, polyvinyl alcohol, polyacrylonitrile, polyamide, polytetrafluorethylene, polyparaphenylene terephthalamide, cellulose, carbon-reinforced polymer composites, shape memory alloys, PEEK, titanium, titanium alloys, cobalt chrome alloys, stainless steel, and combinations thereof.

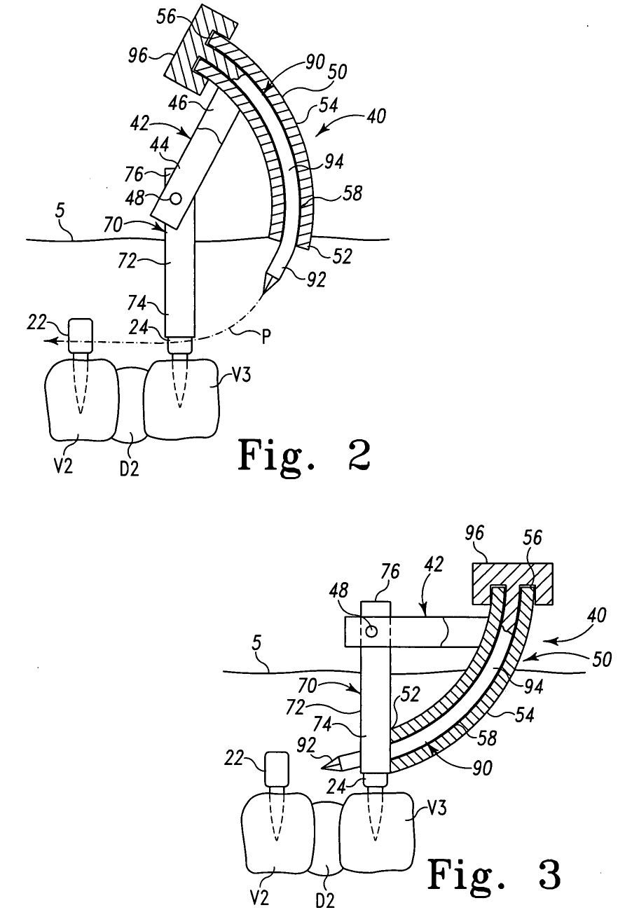

Insertion of dynamic implant 30 into the patient in a minimally invasive or percutaneous procedure can result in the implant becoming misaligned with the desired implantation location during insertion as a result of the leading end of the implant deflecting off tissue or other anatomical elements along the insertion pathway. FIG. 2 shows one embodiment of a guide instrument 40 that is manipulatable within the patient to provide a minimally invasive insertion pathway for dynamic implant 30. Guide instrument 40 includes a first portion 42 mounted to at least one anchor extension 70 and a second portion 50 extending from first portion 42 in a transverse relationship. First portion 42 includes a body forming first and second arms 44, 46 that extend on opposite sides of anchor extension 70 (only a portion of arm 44 is shown.) Other embodiments contemplate first portion 42 with a single arm. Second portion 50 includes an arcuate body 54 extending between distal end 52 and an opposite proximal end 56. Body 54 defines a passage 58 extending between and opening at distal and proximal ends 52, 56. Body 54 can be curved to match or generally follow the radius of curvature defined by an insertion pathway P, as discussed further below. Body 54 can also be tapered distally to distal end 52 to facilitate insertion and dilation of tissue as body 54 is moved through the tissue of the patient.

First and second arms 44, 46 are pivotally coupled adjacent their proximal ends to anchor extension 70 at a pivot connection 48. Pivot connection 48 allows guide instrument 40 to be pivoted about connection 48 relative to anchor extension 70. The pivoting movement of guide instrument 40 allows second portion 50 to be swung along an arc having a rotational center at connection 48 and guided into the patient along an arcuate or curved insertion pathway P. Pathway P extends through skin S of the patient and the bodily tissue between skin S and the vertebrae V2, V3. Other embodiments contemplate that a portion of skin or tissue is removed or retracted to accommodate insertion of second portion 50 to form pathway P.

Connection point 48 is referenced to vertebra V3 by anchor 24 and anchor extension 70 mounted to anchor 24. Anchor extension 70 includes an elongate body 72 extending between a distal end 74 removably mounted to anchor 24 and an opposite proximal end 76. Any suitable connection arrangement between anchor extension 70 and anchor 24 is contemplated, including clamping engagement, threaded engagement, or frictional engagement, for example. The length of first portion 42 between connection 48 and its distal end, from which second portion 50 extends, is sized relative to the length of anchor extension 70 so that the distal end 52 of second portion 50 aligns with anchor 24 when second portion 50 is moved along pathway P to position distal end 52 at a location adjacent to anchor 24, as shown in FIG. 3.

To further facilitate insertion of body 54 and creation of pathway P to anchor 24, a pathway forming instrument 90 is engaged to second portion 50 of guide instrument 40. Pathway forming instrument 90 includes a distal end portion 92 that forms a trocar tip, blunt dissector, or other suitable form to facilitate creation of a pathway through bodily tissue as it passes therethrough. Pathway forming instrument 90 further includes an elongated shaft 94 extending proximally from distal end portion 92 to a proximal connector 96. Shaft 94 extends though passage 58 of body 54 so that proximal connector 96 is secured to second portion 50 at its proximal end 56. Various means for positively engaging proximal connector 96 to guide instrument 40 are contemplated, including threaded connections, twist-type locking arrangements, snap fits, interference fits, and supplemental fasteners, for example.

Shaft 94 extends from the proximal connector 96 to distal end portion 92 and is sufficiently rigidly contained within passage 58 to maintain the alignment of distal end portion 92 as it is forced through the bodily tissue to anchor 24. Shaft 94 includes a curvature to generally parallel the curvature of passage 58 to facilitate insertion and withdrawal of pathway forming instrument 90 in passage 58. In the illustrated embodiment, distal end portion 92 has a length sized to extend through anchor 24 toward anchor 22. In other embodiments, distal end portion 92 has a length sized to extend between anchors 22, 24 such as shown in FIG. 8. Proximal connector 96 is provided with an enlarged end that provides an impactor cap to receive impaction forces to facilitate moving distal end portion 92 through the bodily tissue.

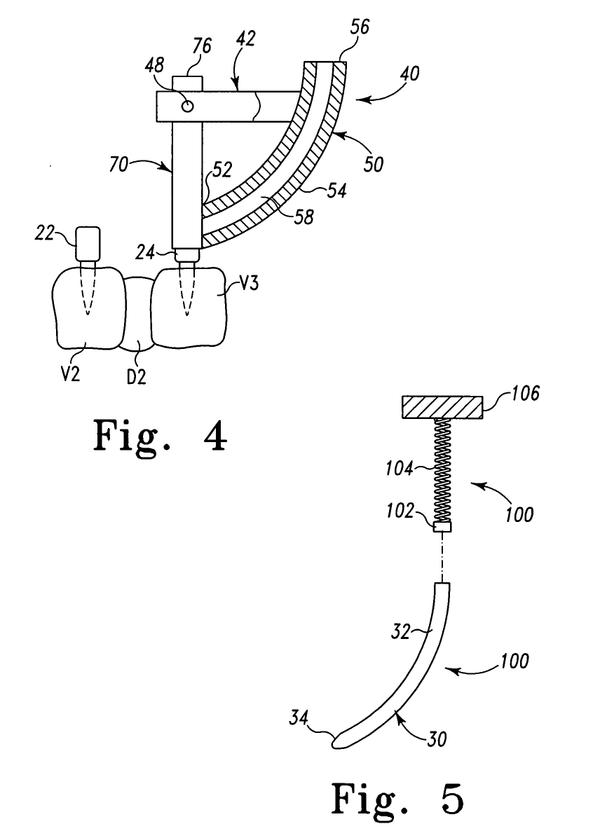

Once the pathway is formed to at least anchor 24 with the instrument assembly including guide instrument 40 and pathway forming instrument 90, pathway forming instrument 90 is removed from passage 58 of guide instrument 40 as shown in FIG. 4. Passage 58 forms an insertion path along which dynamic implant 30 can be moved from outside the patient to a location adjacent to anchors 22, 24 without encountering resistance from tissue that may misalign or misdirect implant 30 as it is inserted. In FIG. 5, there is shown an inserter instrument 100 including a distal portion 102 and an elongated shaft 104 extending from distal portion 102 to a proximal portion 106. Proximal portion 106 includes a cap, knob or other gripping element to facilitate grasping of inserter instrument 100. In one embodiment, shaft 104 is formed with a curvature that generally parallels the curvature of passage 58. In another embodiment, shaft 104 is flexible to facilitate shaft 104 moving along an arcuate insertion path defined by passage 58 and to direct dynamic implant 30 along the same. For example, in one embodiment at least a portion of shaft 104 includes a tightly wound coil spring arrangement. Distal portion 102 includes a connection arrangement to engage a trailing end 32 of dynamic implant 30 in an end-to-end manner with shaft 102. Any suitable connection arrangement between inserter instrument 100 and dynamic implant 30 is contemplated, including threaded engagement, friction fits, clamping arrangements, fasteners, and ball-detent mechanisms, for example.

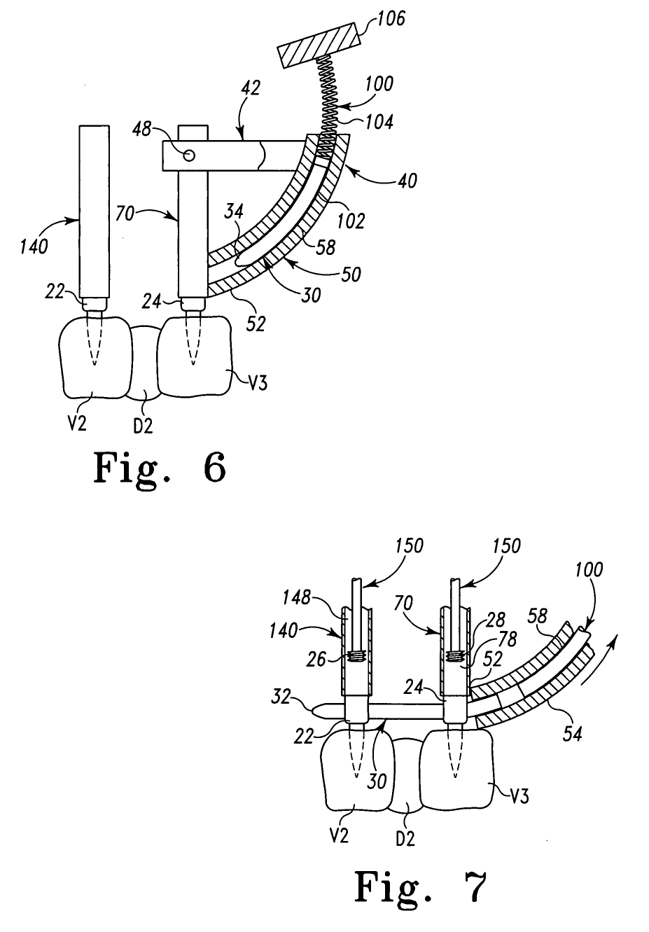

The arcuate shape of body 54 of second portion 50 can contact dynamic implant 30 as it is inserted though passage 58 and direct the leading end 34 of dynamic implant 30 toward distal end 52 for delivery through the distal end opening of passage 58 to anchor 24 as shown in FIG. 6. Leading end 34 of dynamic implant 30 is placed into passage 58 and inserter instrument 100 is manipulated to push dynamic implant 30 through passage 58 and deliver it through the opening at distal end 52. Dynamic implant 30 is inserted into a passage or receptacle of anchor 24, and then pushed from anchor 24 to anchor 22, as shown in FIG. 7, to extend between and connect anchors 22, 24 to one another and provide dynamic stabilization of the motion segment including vertebrae V2, V3.

With dynamic implant 30 extending between anchors 22, 24, dynamic implant 30 is secured to anchors 22, 24 with respective ones of the engaging member 26, 28, respectively, as shown in FIG. 7. Also shown in FIGS. 6 and 7 is a second anchor extension 140 is engaged to anchor 22 to provide a passage 148 for delivery and/or securing of engaging member 26 to anchor 22 with driver instrument 150. Anchor extension 70 similarly includes a passage 78 to receive engaging member 28 and driver instrument 150 to deliver and/or secure engaging member 28 to anchor 24. After engagement of dynamic implant 30 to anchors 22, 24, inserter instrument 100 is released from dynamic implant 30 and can be withdrawn from passage 58, although withdrawal is not necessary. Guide instrument 40 is moved back along the insertion path P to remove second portion 50 from the patient, and then uncoupled from anchor extension 70. Anchors extensions 70, 140 are uncoupled from the respective anchors 24, 22 and the insertion procedure is completed.

Referring now to FIG. 8, there is shown another embodiment guide instrument 40′ that is similar to guide instrument 40 discussed above. Guide instrument 40′ includes another embodiment first portion 42′ that is configured to be pivotally engaged to both anchor extensions 70, 140 adjacent their proximal ends at connection 48′. First portion 42′ further includes first and second arms 44′, 46′ extending from connection 48′ to second portion 50. Anchors extensions 70, 140 are movable relative to vertebrae V2, V3 so that the proximal ends can be positioned adjacent one another while their distal ends are engaged to the respective anchor 22, 24. In other embodiments, first portion 42′ includes a single arm pivotally coupled between anchor extensions 70, 140.

In FIG. 8 guide instrument 40′ is shown with another embodiment pathway forming instrument 90′. Pathway forming instrument 90′ is similar to instrument 90 discussed above, but includes a distal end portion 92′ having a length extending from distal end 52 of second portion 50 that is sized to extend between anchors 22, 24.

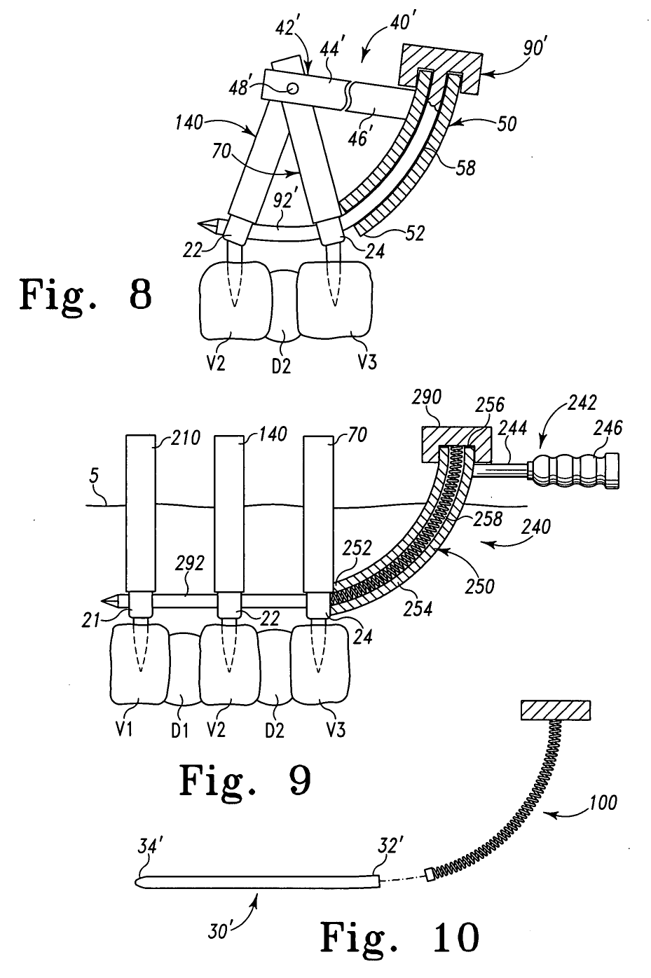

Referring now to FIG. 9, there is shown another embodiment guide instrument 240. Guide instrument 240 includes a first portion 242 extending from a second portion 250. Second portion 250 is similar to second portion 50 discussed above. Second portion 250 includes a body 254 extending between a distal end 252 and a proximal end 256. Body 254 defines a passage 258 extending between and opening at distal end 252 and proximal end 256. Body 254 and passage 258 are shown to be arcuate between distal end 252 and proximal end 256 in a manner similar to body 54.

First portion 242 includes an arm 244 extending from proximal end 256 of second portion 250 in a generally linear and transverse relationship to body 254. Arm 244 includes a handle 245 associated therewith so that guide instrument 240 can be grasped and manipulated by the surgeon during insertion through bodily tissue. Guide instrument 240 further includes pathway forming instrument 290 positioned in passage 258 of second portion 250. Pathway forming instrument 290 is similar to pathway forming instrument 90 discussed above, but includes a distal portion 292 extending from distal end 252 having a length sized to extend between three anchors 21, 22, 24 engaged to respective ones of vertebrae V1, V2, V3. Anchors 21, 22, and 24 can further include respective ones of three anchor extensions 210, 140, and 70 engaged thereto and extending proximally therefrom so that the proximal ends of the anchor extensions 210, 140, 70 are accessible to manipulate the anchors and to deliver or provide access to engaging members to secure dynamic implant 30′ to the anchors 21, 22, 24.

In use, guide instrument 240 is not mounted to anchor extensions 70, 140, 210. Guide instrument 240 and pathway forming instrument 290 are assembled and inserted together into the patient and guided and manipulate percutaneously through skin and/or tissue toward anchor 24. Guide instrument 240 is manipulated to extend distal end portion 292 between anchors 21, 22 and 24, using freehand techniques aided by image guided navigation and tactile feedback to the surgeon. The insertion point into skin S and pathway for insertion can be planned pre-operatively via imaging of the spinal motion segment to be treated and then tracked real time during surgery with the image guided navigation system. After forming a pathway between anchors 21, 22, 24 with pathway forming instrument 290, pathway forming instrument 290 is withdrawn from passage 258 while second portion 250 is maintained in position with distal end 252 adjacent to anchor 24. Dynamic implant 30′ has a length between leading end 34′ and trailing end 32′ sized to extend between anchors 21, 22 and 24. Trailing end 32′ of dynamic implant 30′ is connected with inserter instrument 100 (FIG. 10) and delivered with leading end 32′ passing through anchors 24 and 22 to anchor 21 using inserter instrument 100 through passage 258 of guide instrument 240. Dynamic implant 30′ is secured to anchors 21, 22, 24 with engaging members engaged to the respective anchors 21, 22, 24. Extensions 70, 140, 210 and guide instrument 240 can then be withdrawn from the patient to complete the procedure.

Further examples of suitable anchor extensions, engagement relationships between anchor extensions and anchors, techniques for coupling the guide instrument to the anchor extensions, and freehand techniques for positioning an instrument adjacent to the anchor with image guided navigation are discussed in U.S. Patent Application Publication No. 2002/0161368 published on Oct. 31, 2002; U.S. patent application Ser. No. 11/213,473 filed on Aug. 26, 2005; and U.S. patent application Ser. No. 11/348,999 filed on Feb. 7, 2006; each of which is incorporated herein by reference in its entirety.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. A instrument assembly for delivering a dynamic implant in bodily tissue, comprising:
at least one anchor engageable to a bony element;
at least one anchor extension including an elongated body extending from a distal end engageable to said anchor to an opposite proximal end;
a guide instrument movably mounted to said at least one anchor extension adjacent said proximal end of said at least one anchor extension, wherein said guide instrument includes a first portion extending from said anchor extension to a second portion extending transversely to said first portion, said second portion including an elongated body defining a passage extending therethrough between a distal end of said second portion and a proximal end of said second portion;
a pathway forming instrument removably engaged to said second portion, wherein said pathway forming instrument includes an end portion configured to pass through bodily tissue extending from said distal end of said second portion and a shaft portion extending from said end portion through said passage of said second portion to said proximal end of said second portion; and
an inserter instrument positionable in said passage of said second portion when said pathway forming instrument is removed from said passage, wherein said inserter instrument includes a shaft with a proximal end and an opposite distal end engageable to the dynamic implant, said inserter being operable to move the dynamic implant through said passage from said proximal end of said second portion through said distal end of said second portion.

2. The assembly of claim 1, wherein said at least one anchor extension includes two anchor extensions, and further comprising a second anchor engageable to a second bony element and said anchor extensions are engageable to a respective one of said anchors.

3. The assembly of claim 2, wherein said first portion of said guide instrument is pivotally engageable to said two anchor extensions adjacent proximal ends of said two anchor extensions.

4. The assembly of claim 3, wherein said second portion of said guide instrument extends along an arc between said distal and proximal ends of said second portion.

5. The assembly of claim 4, wherein said passage in said second portion extends along said arc.

6. The assembly of claim 5, wherein said shaft of said inserter instrument is flexible to bend along said arc.

7. The assembly of claim 6, wherein said shaft of inserter instrument is a coil spring.

8. The assembly of claim 1, wherein said shaft portion of said pathway forming instrument includes an impactor cap at a proximal end thereof.

9. The assembly of claim 1, wherein said at least one anchor extension defines a passage extending between said distal and proximal ends thereof, said at least one anchor being accessible through said passage to secure an engaging member to said at least one anchor to secure the dynamic implant to said at least one anchor.

10. The assembly of claim 1, wherein said pathway forming instrument includes a proximal end member removably engageable to said proximal end of said second portion of said guide instrument.

11. The assembly of claim 1, wherein said second portion and said passage of said second portion are curved between said distal and proximal ends of said second portion.

12. The assembly of claim 11, wherein said shaft portion of said pathway forming instrument and said shaft of said inserter instrument are each curved along a length thereof.

13. An instrument assembly for delivering a dynamic implant in bodily tissue, comprising:
a guide instrument including a first portion extending from a first end to an opposite second end and a second portion including an elongated body extending from said second end of said first portion and transversely to said first portion to a distal delivery end remote from said second end of said first portion, said second portion further including a passage defined by said body and opening at said distal delivery end and at a proximal end of said second portion;
a pathway forming instrument extending in said passage of said second portion, said pathway forming instrument including an end portion projecting from said distal delivery end of said second portion, said end portion being configured to pass through bodily tissue to an implantation location, said pathway forming instrument further including a shaft extending from said end portion through said passage of said second portion to a proximal end of said pathway forming instrument engageable to said second portion of said guide instrument; and
an inserter instrument including a proximal end, an elongate shaft extending from said proximal end, and a distal connecting end removably engageable to the dynamic implant, wherein when said pathway forming instrument is removed from said second portion said inserter instrument is positionable into said passage of said second portion to deliver the dynamic implant through said distal delivery end to the implantation location.

14. The assembly of claim 13, further comprising at least one anchor extension engageable to an anchor engageable to a bony element in the patient, said at least one anchor extension including an elongate body extending between a distal end removably engageable to the anchor and an opposite proximal end.

15. The assembly of claim 14, wherein said first portion is pivotally engageable to said at least one anchor extension adjacent said proximal end of said at least one anchor extension.

16. The assembly of 15, further comprising a second anchor extension including an elongate body extending from a distal end removably engageable to a second anchor engageable to a second bony element, said elongate body extending from said distal end thereof to a proximal end opposite said distal end, wherein said first portion of said guide instrument is pivotally engageable said proximal ends of both said anchor extensions.

17. The assembly of claim 16, wherein said second portion of said guide instrument extends along an arc between said distal delivery end and said proximal end thereof, said second portion being movable along an arcuate path through bodily tissue by pivoting said first portion about said proximal ends of said anchor extensions.

18. The assembly of claim 13, wherein said second portion and said passage of said second portion are curved along a length thereof between said distal delivery end and said proximal end thereof.

19. The assembly of claim 13, wherein said first end of said first portion of said guide instrument includes a handle.

20. The assembly of claim 13, wherein said proximal end of said pathway forming instrument includes an impaction member.

21. The assembly of claim 13, wherein said second portion of said guide instrument tapers from said proximal end to said distal delivery end.

22. A method for inserting a dynamic implant in a minimally invasive procedure, comprising:
engaging an anchor to a bony element;
engaging an elongated extension to the anchor with the elongated extension extending from the anchor to a proximal end;
providing a guide instrument including a first portion for manipulating the guide instrument and an elongated second portion extending transversely to the first portion;
advancing the second portion of the guide instrument through bodily tissue to a location adjacent the anchor;
moving the dynamic implant through a passage of the second portion from a location outside the bodily tissue to the location adjacent the anchor; and
engaging the dynamic implant to the anchor.

23. The method of claim 22, wherein advancing the second portion includes forming a pathway through the bodily tissue to the anchor with a pathway forming instrument extending distally from the second portion of the guide instrument.

24. The method of claim 23, wherein the pathway forming instrument extends through the passage of the second portion, and further comprising removing the pathway forming instrument from the passage before moving the dynamic implant through the passage.

25. The method of claim 24, further comprising securing the dynamic implant to a distal end of an inserter instrument before moving the dynamic implant through the passage and moving the dynamic implant through the passage includes advancing the dynamic implant through a distal end opening of the second portion and between first and second anchors engaged to respective ones of first and second bony elements.

26. The method of claim 25, further comprising bending a shaft of the inserter instrument in the passage of the second portion while moving the dynamic implant through the passage.

27. The method of claim 22, further comprising engaging a second anchor to a second bony element and advancing the second portion of the guide instrument includes advancing a pathway forming instrument along a non-linear path through bodily tissue to the anchor engaged to the bony element and then to the second anchor engaged to the second bony element.

28. The method of claim 27, wherein moving the dynamic implant includes moving the dynamic implant along the non-linear path and between the anchors and engaging the dynamic implant includes engaging the dynamic implant to both anchors.

29. The method of claim 28, further comprising engaging a second elongate anchor extension to the second anchor.

30. The method of claim 29, further comprising pivotally engaging the guide instrument to the elongated anchor extensions.

31. The method of claim 22, further comprising pivotally engaging the guide instrument to the proximal end of the anchor extension.
